(19) United States
(12) Patent Application Publication
(10) Pub. No.: US 2013/0131527 A1
(43) Pub. Date: May 23, 2013

(54) METHOD FOR GUIDING AND MONITORING INTRAPERICARDIAL LEAD POSITION FOR AN INTRAPERICARDIAL LEAD SYSTEM

(71) Applicants: Xiaoyi Min, Thousand Oaks, CA (US); Anna Raskin, San Franciso, CA (US); Kevin L. Morgan, Simi Valley, CA (US); Stephanie Toy, Santa Clarita, CA (US); Joyce Tao, Arcadia, CA (US)

(72) Inventors: Xiaoyi Min, Thousand Oaks, CA (US); Anna Raskin, San Franciso, CA (US); Kevin L. Morgan, Simi Valley, CA (US); Stephanie Toy, Santa Clarita, CA (US); Joyce Tao, Arcadia, CA (US)

(73) Assignee: PACESETTER, INC., Sylmar, CA (US)

(21) Appl. No.: 13/301,500

(22) Filed: Nov. 21, 2011

(51) Int. Cl. A61B 5/0408

(52) U.S. Cl. 600/510

(57) ABSTRACT

A first cardiac signal associated with an activity of a first implant site of a heart during a cardiac cycle is sensed. A second cardiac signal is sensed using an intrapericardial lead located on an epicardial surface proximate a second implant site of the heart. The second cardiac signal is associated with an activity of the second implant site during the cardiac cycle. A timing delay between the activity of the first implant site and the activity of the second implant site is obtained and analyzed to determine if the intrapericardial lead location is appropriate. The preceding is repeated until an appropriate intrapericardial lead location is determined. Other measurements obtained during implant determine whether the intrapericardial lead location is at or near slow conduction zone and whether the intrapericardial lead is placed at the location having the greatest mechanical delay. Post implant measurements determine whether the intrapericardial lead has migrated.

### FIELD

The present disclosure is related, generally, to placing a lead of an implantable medical device and, more specifically to guiding and monitoring the position of a lead configured for placement in the pericardial space of a heart.

### BACKGROUND

The heart is a muscular organ having multiple chambers that operate in concert to circulate blood throughout the body's circulatory system. To circulate blood throughout the body's circulatory system, a beating heart performs a cardiac cycle. The contractions of the muscular walls of each chamber of the heart are controlled by a complex conduction system that propagates electrical signals to the heart muscle tissue to effectuate the atrial and ventricular contractions necessary to circulate the blood. The complex conduction system includes an atrial node (e.g., the sinoatrial node) and a ventricular node (e.g., the atrioventricular node). The sinoatrial node initiates an electrical impulse that spreads through the muscle tissues of the right and left atriums and the atrioventricular node. As a result, the right and left atriums contract to pump blood into the right and left ventricles.

At the atrioventricular node, the electrical signal is momentarily delayed before propagating through the right and left ventricles. Within the right and left ventricles, the conduction system includes right and left bundle branches that extend from the atrioventricular node via a Bundle of His. The electrical impulse spreads through the muscle tissues of the right and left ventricles via the right and left bundle branches respectively. As a result, the right and left ventricles contract to pump blood throughout the body.

Normally, the muscular walls of each chamber of the heart contract synchronously in a precise sequence to efficiently circulate the blood as described above. In particular, both the right and left atriums contract (e.g., atrial contractions) and relax synchronously. Shortly after the atrial contractions, both the right and left ventricles contract (e.g., ventricular contractions) and relax synchronously. Several disorders or arrhythmias of the heart can prevent the heart from operating normally, such as, blockage of the conduction system, heart disease (e.g., coronary artery disease), abnormal heart valve function, or heart failure.

Impaired cardiac performance can result from several abnormalities. Such abnormalities include alterations in the normal electrical conduction patterns and mechanical abnormalities in myocardial contractility. For example, blockage in the conduction system can cause a slight or severe delay in the electrical impulses propagating through the atrioventricular node, causing inadequate ventricular relaxation and filling. In situations where the blockage is in the ventricles (e.g., the right and left bundle branches), the right and/or left ventricles can only be excited through slow muscle tissue conduction. As a result, the muscular walls of the affected ventricle do not contract synchronously (e.g., asynchronous contraction), thereby, reducing the overall effectiveness of the heart to pump oxygen-rich blood throughout the body.

Various medical procedures have been developed to address these and other heart disorders. In particular, cardiac resynchronization therapy (“CRT”) can improve the conduction pattern and sequence of the heart. CRT involves the use of an artificial electrical stimulator that is surgically implanted within the patient's body. Leads from the stimulator can be affixed at a desired location within the heart to effectuate synchronous atrial and/or ventricular contractions. Typically, the location of the leads (e.g., stimulation site) is selected based upon the severity and/or location of the blockage. Electrical stimulation signals can be delivered to resynchronize the heart, thereby, improving cardiac performance.

Even with technology advances in CRT, 30% of patients still do not respond to biventricular (BIV) pacing therapy. It is known that left ventricle positioning of a lead is a critical factor to improve CRT response. However, intracardiac left ventricle leads have limited access to left ventricle locations. In addition, implant of the left ventricle pacing lead in areas with impaired tissue may result in less than optimal cardiac resynchronization. Therefore, there is a need for CRT methods and devices that can constantly and/or automatically optimize CRT for a patient based on lead positioning.

### SUMMARY

According to aspects of the present disclosure, a method for guiding and/or monitoring a location of an intrapericardial lead includes sensing a first cardiac signal associated with an activity of a first implant site of a heart during a cardiac cycle; sensing a second cardiac signal from the intrapericardial lead located on an epicardial surface proximate a second implant site of the heart, the second cardiac signal associated with an activity of the second implant site of the heart during the cardiac cycle; obtaining a timing delay between the activity of the first implant site and the activity of the second implant site; and analyzing the location of the intrapericardial lead based on the timing delay; and repeating the preceding steps until an appropriate intrapericardial lead location is determined.

In a detailed aspect, the activity of the first implant sight is a cardiac polarization, the activity of the second implant sight is a cardiac polarization and analyzing comprises comparing the timing delay, which in this case is an electrical separation, to a threshold indicative of an appropriate intrapericardial lead location. The electrical separation may be based on measured atrioventricular delays, intra-ventricular delays or inter-ventricular delays associated with the intrapericardial lead.

In other aspects, pacing latency and/or evoked response at the intrapericardial lead are measured to determine if the intrapericardial lead location is at or near a slow conduction area such as an ischemic region or myocardial infarct zone. Slow conduction areas may also be detected based on ST segment measurements obtained from cardiac electrograms sensed using the intrapericardial lead.

In yet another aspect, electrical separations, pacing threshold, cardiogenic impedances and electro-mechanical delay are obtained post implant and monitored to detect for sudden changes indicative of intrapericardial lead migration.

The foregoing has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF FIGURES

The features, nature, and advantages of the present disclosure will become more apparent from the detailed description set forth below when taken in conjunction with the drawings in which like reference characters identify correspondingly throughout.

FIG. 1 schematically illustrates an exemplary implantable medical device (IMD) in electrical communication with the heart of a patient.

FIG. 2 schematically illustrates an exemplary implantable stimulation device.

FIG. 3A-3C is a top perspective view of an intrapericardial lead.

FIG. 4 illustrates an exemplary intrapericardial lead location system.

FIG. 5 illustrates an exemplary flowchart of a method for guiding and monitoring intrapericardial lead position.

FIG. 6 illustrates flowchart of a method for guiding and monitoring a location of an intrapericardial lead.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion. The following description includes the best mode presently contemplated for practicing the present teachings. The description is not to be taken in a limiting sense but is merely for the purpose of describing the general principles of the illustrative embodiments. The scope of the present teachings should be ascertained with reference to the claims. In the description that follows, like numerals or reference designators will refer to like parts or elements throughout.

Some portions of the following detailed description are presented in terms of algorithms and symbolic representations of operations on data bits or binary digital signals within a computer memory. These algorithmic descriptions and representations may be the techniques used by those skilled in the data processing arts to convey the substance of their work to others skilled in the art.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Discussions herein utilizing terms such as, for example, “processing”, “computing”, “calculating”, “determining”, “establishing”, “analyzing”, “checking”, “measuring”, “sensing”, or the like, may refer to operation(s) and/or process(es) of a computer, a computing platform, a computing system, or other electronic computing device, that manipulate and/or transform data represented as physical (such as, electronic) quantities within the computer's registers and/or memories into other data similarly represented as physical quantities within the computer's registers and/or memories or other information storage medium that may store instructions to perform operations and/or processes.

The terms “plurality” and “a plurality” as used herein includes, for example, “multiple” or “two or more”. For example, “a plurality of items” includes two or more items.

Unless otherwise noted, or as may be evident from the context of their usage, any terms, abbreviations, acronyms or scientific symbols and notations used herein are to be given their ordinary meaning in the technical discipline to which the disclosure most nearly pertains. The following or above-noted terms, abbreviations and/or acronyms may be used throughout the descriptions presented herein and should generally be given the described meaning(s) unless contradicted or elaborated upon by other descriptions set forth herein. Some of the terms set forth below may be registered trademarks (®).

When terms (such as abbreviations) are used in the description, no distinction should be made between the use of capital (uppercase) and lowercase letters. For example, “ABC”, “abc” and “Abc”, or any other combination of upper and lower case letters with these 3 letters in the same order should be considered to have the same meaning as one another, unless indicated or explicitly stated to be otherwise. The same commonality generally applies to glossary or other introduced terms (such as abbreviations), which include subscripts, which may appear with or without subscripts, such as “Xyz” and “X_{yz}”. Additionally, plurals of glossary or other introduced terms may or may not include an apostrophe before the final “s”—for example, ABCs or ABC's.

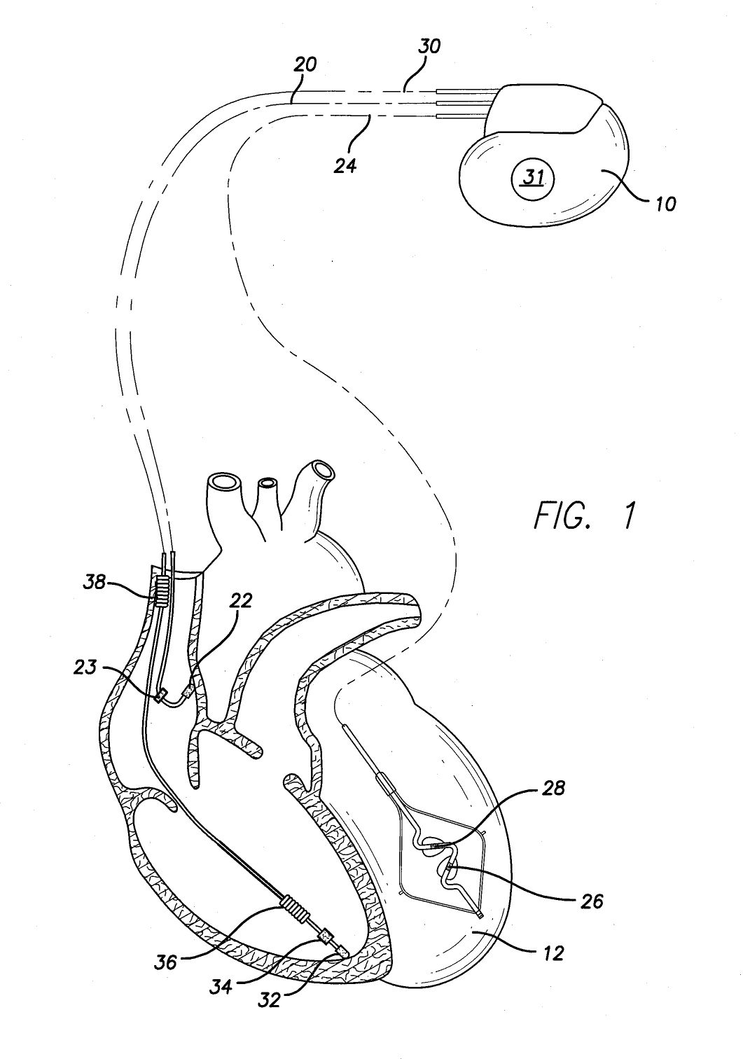

With reference to FIG. 1, an exemplary implantable medical device (IMD) 10 will be described in detail. The IMD 10 is in electrical communication with the heart 12 of a patient by way of three leads, 20, 24 and 30, suitable for delivering multi-chamber stimulation and shock therapy. To sense atrial cardiac signals and to provide right atrial chamber stimulation therapy, the IMD 10 is coupled to an implantable right atrial lead 20 having at least an atrial tip electrode 22, which typically is implanted in the right atrial appendage, and an atrial ring electrode 23.

To sense ventricular cardiac signals and to provide left chamber pacing therapy, the IMD 10 is coupled to an intrapericardial (IP) lead 24 designed for placement on the epicardial surface of the heart in the region of the left ventricle for positioning electrodes adjacent to the left ventricle. An exemplary IP lead 24 is designed to receive ventricular cardiac signals and to deliver left ventricular pacing therapy using at least a left ventricular distal electrode 26 and a left ventricular proximal electrode 28.

The IMD 10 is also shown in electrical communication with the heart by way of an implantable right ventricular lead (RV lead) 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart so as to place the right ventricular tip electrode 32 in the right ventricular apex so the RV coil electrode 36 is positioned in the right ventricle and the SVC coil electrode 38 is positioned in the superior vena cava. Accordingly, the right ventricular lead 30 is capable of receiving cardiac signals, and delivering stimulation in the form of pacing and shock therapy to the right ventricle. An accelerometer 31 can also be provided.

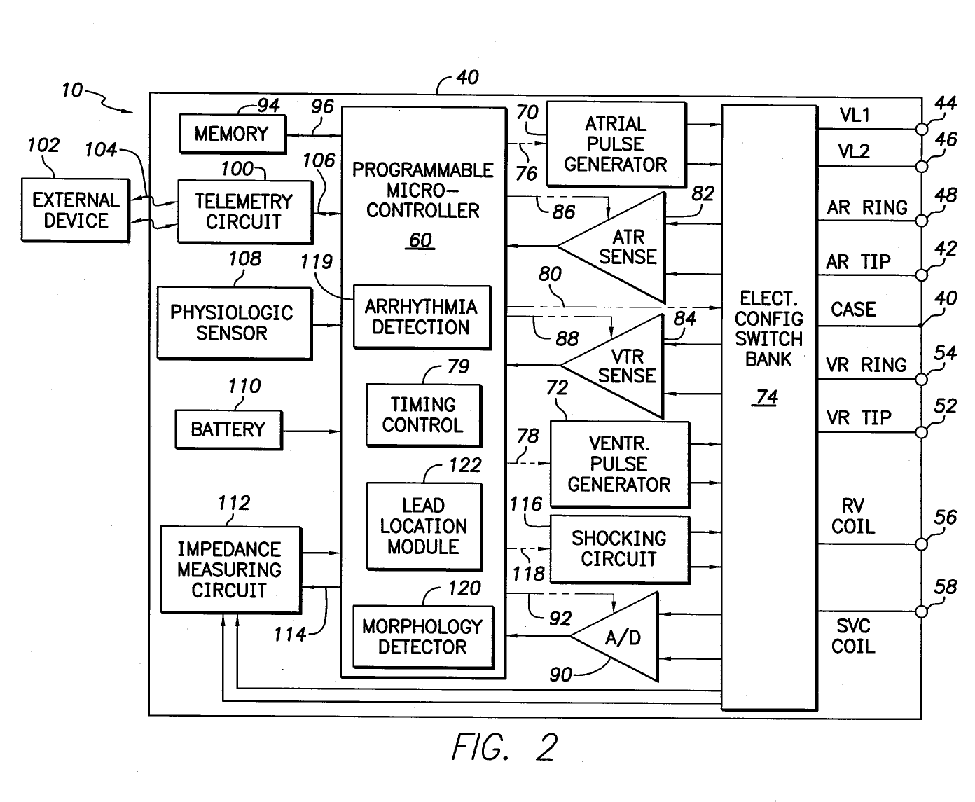

As illustrated in FIG. 2, a simplified block diagram is shown of the multi-chamber implantable medical device (IMD) 10, which is capable of treating both fast and slow arrhythmias with stimulation therapy, including cardioversion, defibrillation, and pacing stimulation. Although FIG. 2 illustrates a detailed view of an implantable medical device, it should be understood that the present disclosure works equally as well with a pacing system analyzer (PSA). The IMD 10 is configured as a system in which the various embodiments of the present teachings may operate. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. In some aspects of the disclosure, PSA, for example, can be used instead of the IMD 10 to guide lead position and determine optimal or improved location of leads based on electrical parameters evaluated during IP lead implant. Leads may be coupled to the PSA such that values for ventricular delays, including for example atrio-ventricular delays, intra-ventricular delays and inter-ventricular delays, can be determined in conjunction with an algorithm configured to determine improved timing or delay, as explained in more detail below.

The housing 40 for the IMD 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes, 36 and 38, for shocking purposes. The housing 40 further includes a connector (not shown) having multiple terminals, 42, 44, 46, 48, 52, 54, 56 and 58 (shown schematically and, for convenience, the names of the electrodes to which they are connected are shown next to the terminals).

To achieve right atrial sensing and pacing, the connector includes at least a right atrial tip terminal (AR TIP) 42 adapted for connection to the atrial tip electrode 22 and a right atrial ring (AR RING) terminal 48 adapted for connection to the right atrial ring electrode 23. To achieve left chamber sensing and pacing, the connector includes at least a left ventricular distal terminal (VL1) 44 and a left ventricular proximal terminal (VL2) 46 which are adapted for connection to the left ventricular distal electrode 26 and the left ventricular proximal electrode 28 respectively. To support right chamber sensing, pacing and shocking, the connector further includes a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32, right ventricular ring electrode 34, the RV coil electrode 36, and the SVC coil electrode 38, respectively.

The IMD 10, pacing system analyzer (PSA) or other component includes a programmable microcontroller 60, which controls the various modes of stimulation therapy. As is well known in the art, the microcontroller 60 (also referred to as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by program code stored in a designated block of the memory. The details of the design and operation of the microcontroller 60 are not critical to the present teachings. Rather, any suitable microcontroller 60 may be used that carries out the functions described. The use of microprocessor-based control circuits for performing timing and data analysis functions is well known in the art.

As shown in FIG. 2, an atrial pulse generator 70 and a ventricular pulse generator 72 generate pacing stimulation pulses for delivery by the right atrial lead 20, the right ventricular lead 30, and/or the coronary sinus lead 24 via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators or shared pulse generators. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 that controls the timing of such stimulation pulses (such as pacing rate, atrio-ventricular (AV) delay, or ventricular interconduction (V-V) delay, and the like) as well as to keep track of the timing of refractory periods, blanking intervals, noise detection windows, evoked response windows, alert intervals, marker channel timing, etc., as is well known in the art. A switch 74 includes multiple switches for connecting the desired electrodes to the appropriate I/O circuits, thereby providing complete electrode programmability. Accordingly, the switch 74, in response to a control signal 80 from the microcontroller 60, determines the polarity of the stimulation pulses (such as unipolar, bipolar, combipolar, etc.) by selectively closing the appropriate combination of switches (not shown) as is known in the art.

Atrial sensing circuits 82 and ventricular sensing circuits 84 may also be selectively coupled to the right atrial lead 20, the intrapericardial lead 24, and the right ventricular lead 30, through the switch 74 for detecting the presence of cardiac activity in each of the four chambers of the heart. Accordingly, the atrial and ventricular sensing circuits, 82 and 84, may include dedicated sense amplifiers, multiplexed amplifiers or shared amplifiers and may receive control signals 86, 88 from the controller 60. The switch 74 determines the “sensing polarity” of the cardiac signal by selectively closing the appropriate switches, as is also known in the art. In this way, the clinician may program the sensing polarity independent of the stimulation polarity. Each sensing circuit, 82 and 84, employs one or more low power, precision amplifiers with programmable gain and/or automatic gain control, band pass filtering, and a threshold detection circuit, as known in the art, to selectively sense the cardiac signal of interest. An automatic gain control enables the device 10 to effectively address the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (for example: P-waves, R-waves, and depolarization signals associated with fibrillation which are sometimes referred to as “F-waves” or “Fib-waves”) are then classified by the microcontroller 60 by comparing them to a predefined rate zone limit (for example: bradycardia, normal, low rate VT, high rate VT, and fibrillation rate zones) and various other characteristics (for example: sudden onset, stability, physiologic sensors, and morphology, and the like) in order to determine the type of remedial therapy that is needed (for example: bradycardia pacing, anti-tachycardia pacing, cardioversion shocks or defibrillation shocks, and the like).

Cardiac signals are also applied to the inputs of an analog-to-digital (A/D) data acquisition system 90. The data acquisition system 90 is configured to acquire intra-cardiac electrogram (IEGM) signals, convert the raw analog data into a digital signal, and store the digital signals for later processing and/or telemetric transmission to an external device 102. The data acquisition system 90 is coupled to the right atrial lead 20, the IP lead 24 (FIG. 1), and the right ventricular lead 30 through the switch 74 to sample cardiac signals across any pair of desired electrodes. The controller 60 controls the data acquisition system via control signals 92.

The microcontroller 60 is further coupled to a memory 94 by a suitable data/address bus 96. The programmable operating parameters used by the microcontroller 60 are stored and modified, as required, in order to customize the operation of the IMD 10 to suit the needs of a particular patient. The memory 94 includes software modules, such as a lead locator, which, when executed or used by the microcontroller 60, provide the operational functions of the implantable IMD 10. Additional operating parameters and code stored on the memory 94 define, for example, pacing pulse amplitude or magnitude, pulse duration, electrode polarity, rate, sensitivity, automatic features, arrhythmia detection criteria, and the amplitude, wave shape and vector of each shocking pulse to be delivered to the patient's heart within each respective tier of therapy. Other pacing parameters include base rate, rest rate and circadian base rate.

Advantageously, the operating parameters of the implantable device 10 may be non-invasively programmed into the memory 94 through a telemetry circuit 100 in telemetric communication with the external device 102, such as a programmer, trans-telephonic transceiver, a diagnostic system analyzer, or even a cellular telephone. The telemetry circuit 100 is activated by the microcontroller by a control signal 108. The telemetry circuit 100 advantageously allows intra-cardiac electrograms and status information relating to the operation of the device 10 (as contained in the microcontroller 60 or memory 94) to be sent to the external device 102 through an established communication link 104. In one embodiment, the IMD 10 further includes a physiologic sensor 108, commonly referred to as a “rate-responsive” sensor because it adjusts pacing stimulation rate according to the exercise state of the patient. However, the physiological sensor 108 may further be used to detect changes in cardiac output, changes in the physiological condition of the heart, or diurnal changes in activity (for example, detecting sleep and wake states). Accordingly, the microcontroller 60 responds by adjusting the various pacing parameters (such as rate, AV Delay, V-V Delay, and others) at which the atrial and ventricular pulse generators, 70 and 72, generate stimulation pulses. While shown as being included within the IMD 10, it is to be understood that the physiologic sensor 108 may also be external to the IMD 10, yet still be implanted within or carried by the patient.

The IMD 10 additionally includes a battery 110, which provides operating power to all of the circuits shown in FIG. 2. For the IMD 10, which employs shocking therapy, the battery 110 is capable of operating at low current drains for long periods of time, and is capable of providing high-current pulses (for example, for capacitor charging) when the patient requires a shock pulse. The battery 110 also has a predictable discharge characteristic so that elective replacement time can be detected. In one embodiment, the device 10 employs lithium/silver vanadium oxide batteries. As further shown in FIG. 2, the device 10 has an impedance measuring circuit 112 enabled by the microcontroller 60 via a control signal 114.

The IMD 10 detects the occurrence of an arrhythmia and automatically applies an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 or more joules), as controlled by the microcontroller 60. Such shocking pulses are applied to the heart 12 through at least two shocking electrodes, and as shown in this embodiment, selected from the RV coil electrode 36, and/or the SVC coil electrode 38. As noted above, the housing 40 may function as an active electrode in combination with the RV coil electrode 36, or as part of a split electrical vector using the SVC coil electrode 38 (for example, by using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (such as corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

The microcontroller 60 includes a morphology detector 120 for tracking various morphological features within electrical cardiac signals, including intervals between polarization events, elevations between polarization events, durations of polarization events and amplitudes of polarization events. The microcontroller 60 also includes an arrhythmia detection control 119 that analyzes the sensed electrical signals to determine whether or not arrhythmia is being experienced. A lead location module 122, in cooperation with the memory 94, assists in monitoring lead location.

The remaining figures, flow charts, graphs and other diagrams illustrate the operation and novel features of the IMD 10 as configured in accordance with exemplary embodiments of the present teachings. In the flow chart, the various process steps are summarized in individual “blocks.” Such blocks describe specific actions or decisions made or carried out as the process proceeds. Where a microcontroller (or equivalent) is employed, the flow chart provides the basis for heart sound processing that may be used by such a microcontroller (or equivalent IMD controller) to adaptively determine accurate heart sounds. Those skilled in the art may readily write such a program based on the flow chart and other descriptions presented herein.

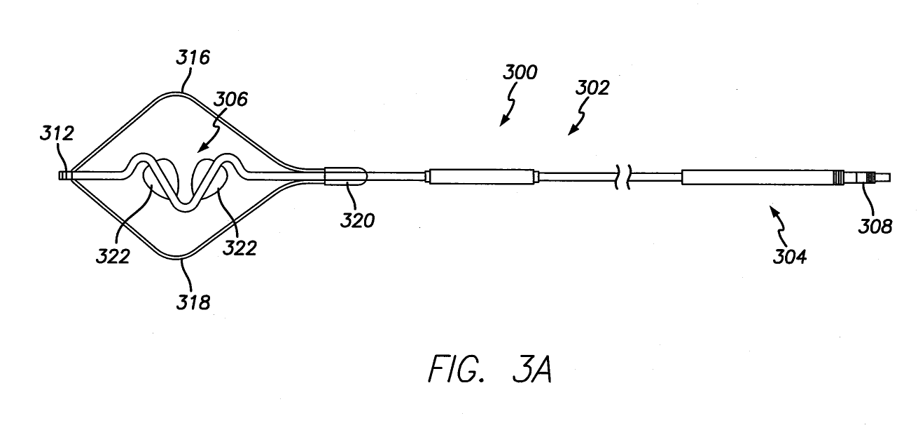

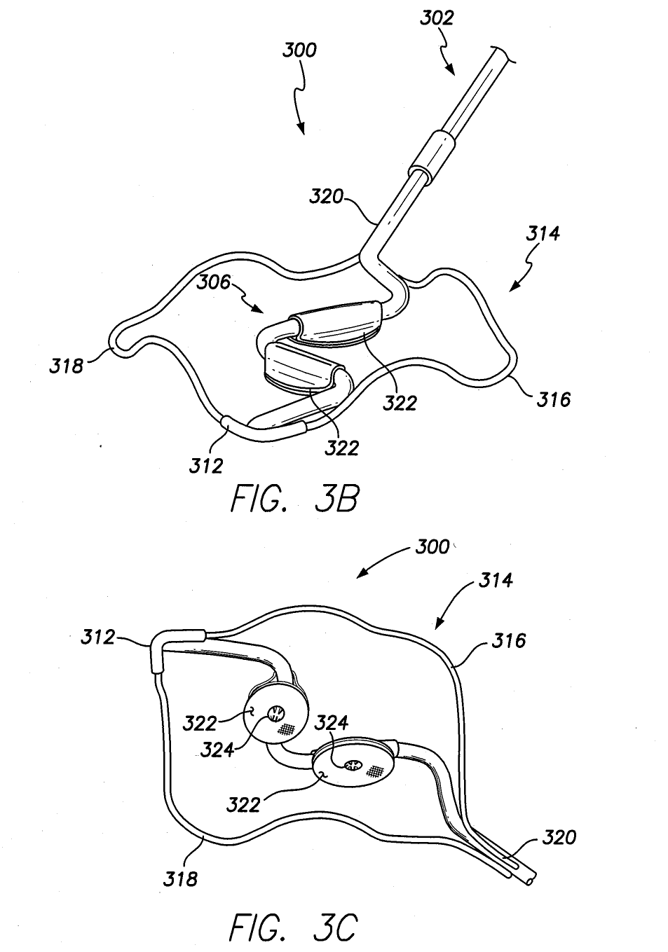

FIGS. 3A, 3B and 3C illustrate exemplary embodiments of intrapericardial leads in accordance with some aspects of the disclosure. As shown in these figures, there is provided an intrapericardial lead 300 including a lead body 302 having a proximal portion 304 and a pre-curved distal end portion 306. The proximal portion 304 may include an electrical connector assembly 308 adapted to be coupled to the pacemaker or implantable medical device 10 or to any other external or internal heart monitoring device. In some aspects, the intrapericardial lead can be implemented according to the teachings of U.S. Pat. No. 7,899,555, issued Mar. 1, 2011, to Morgan et al. the disclosure of which is expressly incorporated by reference herein in its entirety.

The pre-curved distal end portion 306 of the lead body includes a distal tip 312 and carries two three dimensional wings 314 where the distal tip 312 is configured such that the three dimensional wings 314 can recover to its relaxed pre-curved loop configuration upon being released from an extended or elongated configuration (not shown) or in a generally straightened configuration shown in FIG. 3A.

It will be apparent, however, to those skilled in the art that the distal tip 312 may have an aperture in communication with a longitudinally-extending lumen within the lead body 302 to permit delivery of the lead by means of a steerable introducer.

As best seen in FIGS. 3A and 3B, the three dimensional wings 314 can include two three dimensional wire wings or loop portions 316 and 318 that are positioned on opposite sides of the distal end portion 306. The inside of the three dimensional wing structure 314 defines a central region.

As stated above, the distal end portion 306 of the lead body 302 normally assumes a curved, sinuous configuration when it is not urged into its generally straightened configuration. The sinuous configuration extends from the distal tip 312 to a proximal end 320 of the distal end portion and may take various curved forms in different lead embodiments. The three dimensional wings 314, in plan view, may take various forms. For example, the embodiment shown in FIGS. 3A-3C has a generally diamond-shaped configuration.

The distal end portion 306 of the lead body carries at least one passively fixed or anchored electrode assemblies 322 within the confines of the three dimensional wings 314. Each electrode assembly 322 may have an electrode 324. In some aspects of the disclosure, each electrode 324 may carry a plurality of prongs (not shown) that project beyond a flat surface of the electrode assembly 322. The prongs may serve to grip the pericardial tissue and to concentrate the electrical current density, for example.

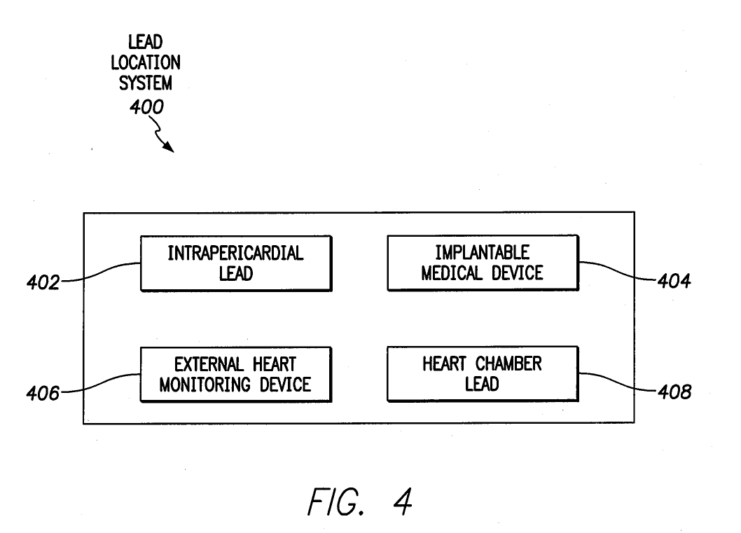

FIG. 4 illustrates an exemplary intrapericardial lead location system according to some aspects of the disclosure. The intrapericardial lead location system 400 may include an intrapericardial lead 402, an implantable medical device 404 that is similar to the implantable medical device 10, an external heart monitoring device 406, such as a PSA, and a heart chamber lead 408.

In some aspects, the intrapericardial lead location system 400 may be configured to function with either the implantable medical device 404, and/or the external heart monitoring device 406. The external heart monitoring device 406 may be a programmed device employed during implantation of the device 404, in which the programmed device can function as a pacemaker, pacing system analyzer (PSA) and/or an electrocardiographic device. In some aspects, the intrapericardial lead 402 may be coupled directly to the implantable medical device 404 post implant, for example, directly to the external heart monitoring device 406 during IMD implant procedures, for example, and/or indirectly to the external heart monitoring device 406 via the wireless communication capability of the implantable medical device 404 during post implant patient follow-up procedures, for example. The heart chamber lead 408 may be an endocardial lead configured for pacing the heart and may be coupled to a pacemaker. While shown as being included within the intrapericardial lead location system 400, it is to be understood that the heart chamber lead 408 may not be incorporated in the system and an external electrocardiographic (EKG) device, for example, may provide the relevant data. The heart chamber lead 408 can be positioned in the right ventricle of the heart or any other chamber of the heart. When the heart chamber lead 408 is positioned in the right ventricle, it may be referred to as the right ventricular lead, e.g., RV lead 30. In some aspects, the heart chamber lead 408 may be coupled to the implantable medical device 404 and/or the external heart monitoring device 406.

The intrapericardial lead 402 may be a passive fixation, bipolar, IS-1 compatible, left ventricular pacing lead. The intrapericardial lead 402 may utilize non-surgical techniques to access the epicardial surface of the heart. Once in the intrapericardial space, navigation of the lead can be obtained by way of a steerable introducer, for example. By using the steerable introducer, access can be obtained to substantially any position on the epicardial surface of the heart, for example, to deliver pacing therapy. In some aspects, the intrapericardial lead 402 may be configured to engage an epicardial surface that is proximate the left ventricle of the heart. In some aspects, the intrapericardial lead 402 may be configured to perform at least some of the functions in coordination with the implantable medical device 404. For example, the intrapericardial lead 402 may be configured to deliver multi-chamber stimulation and shock therapy (e.g., electrical impulses or stimuli) and to sense atrial and ventricular cardiac signals.

In some aspects of the disclosure, the lead location system 400 may incorporate an intracardiac electrogram (IEGM) guided method that is based on atrio-ventricular (AV) delays, intra-ventricular delays and/or inter-ventricular delays in order to guide the intrapericardial lead location during implant (with an external heart monitor like a PSA) and/or to monitor heart failure progression and/or lead location (with an IMD) after implant. The atrio-ventricular, intra-ventricular and/or inter-ventricular delays may be calculated or determined based on information or data (e.g., signals such as cardiac electrogram signals and/or mechanical movements via micro-electromechanical systems (MEMS) sensors, for example) indicative of a patient's heart movements. The information can be recorded, received, measured or sensed by the heart chamber lead 408 (e.g., right ventricular lead 30), the intrapericardial lead 402 (similar to intrapericardial lead 300), the external heart monitoring device 406, and/or an implantable pulse generator (ICD). In general, the conductive system of the heart is organized so that transmission of electrical impulses or stimuli is slightly delayed at the atrio-ventricular node, thus allowing time for the atria to empty their contents into the ventricles before the ventricles begin to contract. In mechanical terms, the delay interval between a right atrial contraction and a right ventricular contraction of the heart may be referred to as an atrio-ventricular delay. In electrical sensing terms, the atrio-ventricular delay corresponds to the difference in time between an atrial cardiac event (either intrinsic/sensed or evoked/paced) and a sensed ventricular cardiac event. The delay with transmission of the heart's electrical impulses or stimuli within a ventricle is referred to as intra-ventricular delay. In mechanical terms, the intra-ventricular delay for the lefty ventricle is the difference is motion of the septal wall and motion of the left ventricular free wall. In electrical sensing terms, the intra-ventricular delay may be defined, for example, as the time between a cardiac polarization in a signal sensed by the right ventricle lead and corresponding cardiac polarization in a signal sensed by the left ventricle lead. The atrio-ventricular and intra-ventricular delays may be increased or decreased due to an unhealthy chamber or other defects/disorders or failure of the heart.

The external heart monitoring device 406 and/or the implantable medical device 404 may be configured to receive or acquire signals from the intrapericardial lead 402 in conjunction with signals from the heart chamber lead (e.g., right ventricular lead 30). For example, the signals can be received at the lead location module 122 of the microcontroller 60 of the implantable medical device 10 or a similar device at the external heart monitoring device 406. In some aspects, the external heart monitoring device 406 and/or the implantable medical device 404 may receives a first cardiac signal representing activity of a first implant location of the heart and a second cardiac signal sensed at the intrapericardial lead 402 that is engaged to a site on an epicardial surface, which is proximate a second implant location of the heart during a heartbeat or cardiac cycle. The second signal may represent activity of a second implant site of the heart during the heartbeat or cardiac cycle. In some aspects, the first implant site can be the right ventricle and the second implant site can be the left ventricle. The external heart monitoring device 406 or the implantable medical device 404 may be configured and/or the intrapericardial lead 402, may be positioned to measure or calculate atrio-ventricular delay and/or intra-ventricular delay based on the first and second cardiac signals.

According to an aspect of the present disclosure, the external heart monitoring device 406 and/or the implantable medical device 404 may be further configured to analyze the location of the intrapericardial lead 402 based on the delay, between the activity of the first chamber and the activity of the second chamber, for example. During implant, the location of the intrapericardial lead 402 may be determined based on whether the delay meets a threshold value. The threshold value can be a predefined threshold value.

In some aspects, after the intrapericardial lead 402 is advanced into the intrapericardial space at implant and connected to the external heart monitoring device 406, the intrinsic conduction delay, or time delay Δ (or electrical separation), between signals sensed by the right ventricular lead and the intrapericardial lead can be measured as follows:

Δ=AR_{LV}−AR_{RV}, where
AR_{LV} represents the time between a stimulated atrial event and an intrinsic event in the left ventricle,
AR_{RV} represents the time between a stimulated atrial event and an intrinsic event in the right ventricle,
   or

Δ=R_{LV}−R_{RV}, where
R_{LV} represents the time within a cardiac cycle at which an intrinsic event is sensed in the left ventricle,
R_{RV} represents the time within a cardiac cycle at which an intrinsic event is sensed in the right ventricle,

If the delay (Δ) is less than a threshold (for example, 30 ms), different intrapericardial lead location may be suggested based on an implementation of the external heart monitoring device 406 and/or the implantable medical device 404. During intrapericardial lead navigation about the epicardial surface of the left ventricle, for example, when Δ is within a pre-determined range (such as 30 ms<Δ<160 ms), the implementation may indicate that the intrapericardial lead 402 is at a proper or desired location. In some aspects, the implementation may indicate the maximum delay (Δ) (based on a series of intrapericardial location assessments), and suggest an optimal, improved and/or desired intrapericardial lead location with improved signal sensing and/or pacing capabilities.

During the implant, the right ventricular lead, for example, may be implanted prior to the intrapericardial lead 402. In this situation, the IEGM guided method can be applied to assess and guide intrapericardial lead positions or locations as described above. In some aspects, the right ventricular lead may not be implanted prior to the intrapericardial lead 402 and in some instances not implanted at all. In this situation, an electrocardiographic device may be used to acquire electrocardiograph signals serving as a reference timing for timing the delays, for example, the peak QRS on ECG signals can be used as reference points. Vectors associated with electrocardiograph signals may be selected to represent the timing at a targeted right ventricle site, for example, to assess the delay (Δ).

In some aspects, inter-ventricular conduction delay (IVCD) can also be used as a criteria for analyzing the location of the intrapericardial lead 402. In order to improve cardiac output by proper synchronization of consecutive contractions of the septum and left ventricle free wall of a heart, it is desirable to improve the duration of a delay interval between a septal wall contraction and a left ventricular contraction. In one aspect of the present disclosure, the most electrically delayed region of the left ventricle, as well as the most mechanically delayed region of the left ventricle are paced.

In some aspects, an IVCD represents the delay between the pacing of the right ventricle (RV_{pace}) and the sensing at the left ventricle (LVₛₑₙₛₑ). In other aspects, an IVCD represents the delay between pacing of the left ventricle (LV_{pace}) and the sensing at the right ventricle (RVₛₑₙₛₑ). The location of the intrapericardial lead 402 can be analyzed based on a determination of whether the IVCD meets a threshold value. In some aspects, if the IVCD does not meet the threshold value, for example, if IVCD is less than 80 milliseconds (ms), accounting for pacing latency, then another intrapericardial lead location may be desirable and may be suggested based on an implementation at the intrapericardial lead 402, an implantable medical device 404 and/or the external heart monitoring device 406. Pacing latency is the delay from pacing stimulus delivery to evoked response detection at the pacing site.

In some aspects of the disclosure, after positioning the intrapericardial lead 402 according to an electrical-separation time-delay determination as described above the lead location system 400 can be used to determine whether the IP lead has been located at or near an ischemic or infarct region in any of several ways described below. If the IP lead is placed at or near an ischemic or infarct zone, a new intrapericardial lead location could be found or suggested as described above in order to avoid the ischemic or infarct zone.

Pacing latency may provide an indication of ischemic or infarct region. In this configuration, a pacing stimulation pulse is delivered to a pacing site through the IP lead. An evoked response is detected for at the pacing site. A pacing latency is calculated as the difference between the time of delivery of the pacing pulse and the detection of the evoked response. The pacing latency is then compared to a threshold indicative of an ischemic or infarct zone. For example, if the pacing latency is greater than 70 milliseconds (ms) then the area of the heart proximate to that location of the intrapericardial lead 402 may be deemed unhealthy and/or a slow conduction zone, e.g. and ischemic or infarct region.

Evoke response may also provide an indication of ischemic or infarct region. In this configuration, a pacing stimulation pulse is delivered to a pacing site through the IP lead. An evoked response is detected for at the pacing site. A parameter of the evoked response, such as peak amplitude or peak positive slope, is measured. The parameter is then compared to a threshold indicative of an ischemic or infarct zone. For example, if the peak amplitude is less than 5 millivolts (mV) or the positive slope is less than approximately 0.3, then the area of the heart proximate to that location of the intrapericardial lead 402 may be deemed an ischemic or infarct region.

In some aspects of the disclosure, an infarct or slow conduction zone can be detected during acute testing or implant of the intrapericardial lead 402. The slow conduction zone or ischemia zone may be detected between the right ventricular lead or can, for example, and the intrapericardial lead 402 by sensing and/or measuring signals across the intrapericardial lead 402 and the right ventricular lead or can and by measuring the ST segment. The ST segment is the portion of an electrocardiogram between the end of the QRS complex and the beginning of the T wave. Because of injury potential due to the implantation of the intrapericardial lead 402, indicated by an elevation or spike on the T wave, the spike due to injury should be differentiated from those associated with a heart disease such as myocardial ischemia or injury and coronary artery disease. The injury inflicted during acute testing or implant is expected to be small or ignorable because the intrapericardial lead 402 is a passive fixation. Therefore, T wave changes from the injury or pressure on the electrodes may not be notable.

In one arrangement, a cardiac electrogram is sensed between the IP lead and either of the RV lead or the can. The elevation of the ST segment within the cardiac electrogram is compared to a baseline ST segment elevation to obtain an ST segment deviation. The segment deviation is compared to a threshold indicative of an ischemic or infarct zone. Exemplary thresholds are described in U.S. Pat. No. 7,792,572, the disclosure of which is hereby incorporated by reference. If the ST segment deviation exceeds the threshold, it may indicate ischemia or infarct regions between the intrapericardial lead 402 and the right ventricular lead or can. In this instance, a different IP lead location could be attempted, determined or suggested to avoid the slow conduction zones.

The intrapericardial lead 402 may include a sensor for detecting mechanical cardiac activity events, i.e., contractions. For example, an accelerometer can be included in the IP lead and used during implant for measuring mechanical delays and/or electro-mechanical delays at different sites. Mechanical delays may include a measure of the delay between an intrinsic atrial cardiac depolarization and sensing of a mechanical contraction by the IP lead. An electro-mechanical delay may include a measure of the delay between delivery of a pacing stimulation pulse through the RV lead and sensing of a mechanical contraction by the IP lead. An electro-mechanical delay may also include a measure of the delay between delivery of a pacing stimulation pulse through the IP lead and sensing of a mechanical contraction by the IP lead. This later electro-mechanical delay may be used to identify slow conduction or infarct zones as such zones have a greater delay between pacing pulse delivery and mechanical contraction relative to non-infarct zones.

By measuring mechanical delay or electro-mechanical delay in addition to electrical delay at intended IP lead locations, the intrapericardial lead 402 can allow for detection of intra-LV mechanical dyssynchrony around the left ventricle. In some aspects, it is desirable to place the IP lead so as to pace the left ventricle, for example, at the site or location with maximum mechanical delay or electro-mechanical delay to improve synchronization.

Optimization or improvement of placement of the intrapericardial lead 402 can benefit from the characterization of mechanical or electro-mechanical delays for each region of a chamber of the heart (e.g., left ventricle). The characterization is based on the fusion of electrical and mechanical data or information measured or sensed at the intrapericardial lead 402, for example. In some aspects of the disclosure, the correlation of the location of the intrapericardial lead 402 identified or suggested based on the maximum mechanical delay or electro-mechanical delay may be identified as an improved or desired location of the intrapericardial lead 402. In general, even among a majority of heart failure patients, electrical and mechanical delays are expected, but in some patients electrical-mechanical delays at different sites could be diversified so that distribution of electrical delays is different from mechanical delays. Therefore, knowing electrical, mechanical and electrical-mechanical delays is desirable to determine the optimal intrapericardial lead location for achieving improved synchrony.

The intrapericardial lead location can be monitored after implant, e.g., through follow ups or automatically by an implantable or external medical device, for example, to check the stability of the lead and to monitor heart failure progression. The IEGM or electrical separation time delay method and, the electro-mechanical delay and mechanical delay implementations described above may be applied in conjunction with other follow-up techniques, described below, to monitor intrapericardial lead stability and heart failure progression.

The electrical separation, i.e., Δ, as described above, between signals sensed at the right ventricular lead and the intrapericardial lead 402 can be obtained during follow-up or acquired from a device (implantable or external) that automatically monitors the electrical separation on a periodic basis, such as daily. The periodic electrical separations may be compared to determine whether a difference in electrical separation meets a threshold value indicative of a sudden change associated with lead migration. For example, a change of approximately 30 ms between adjacent electrical separation measurements may be indicative of lead migration. More gradual changes in electrical separation measurements, for example, a 30 ms change that occurs over a period of weeks or months, may be indicative of heart defect or heart failure progression.

Pacing threshold may also be monitored to detect for lead migration. In this configuration, a baseline pacing threshold for the IP lead is obtained during implant. Thereafter, an observed pacing threshold for the IP lead is determined. The observed pacing thresholds are monitored to detect for a sudden change indicative of intrapericardial lead migration. In one arrangement, a sudden change corresponds to a difference in pacing thresholds of approximately 1 volt between the baseline pacing threshold and an observed pacing threshold.

Cardiogenic impedance changes during and after implant can be used to detect IP lead migration. In some aspects, the intra-cardiac impedance, during and after implant, can be obtained or calculated by pacing from the intrapericardial lead 402 and sensing from the right ventricular lead. Impedances measured during implant and after implant may be processed and compared to a threshold to determine whether the IP lead migrated. For example, daily stroke impedances (SZ) may be measured, where SZ is the difference between the maximum impedance and the minimum impedance measured during a cardiac cycle. Measurements may be obtained over several cardiac cycles and averaged to provide a daily SZ value. A separate daily impedance (Z) measurement may be obtained, where the measurement is gated or timed to a point of the cardiac cycle, such as the peak of the QRS portion of a cardiac electrogram. These measurements may also be obtained over several cardiac cycles and averaged to provide a daily Z value. Differences between adjacent daily impedance values provide a measure ΔZ. Processing ΔZ along with SZ on a periodic basis, such as daily, provides an indication of lead migration. More specifically, if ΔZ/SZ is >than approximately 20%, lead migration is indicated. In the preceding formula, SZ may be either of the adjacent daily stroke impedance values corresponding to the adjacent daily Z values. A more gradual change in ΔZ/SZ, where the adjacent Z values that result in a ΔZ/SZ that exceeds the 20% threshold are a week or a month apart, may be indicative of heart failure progression.

Electro-mechanical delay may be monitored to detect for lead migration. In this configuration, an electro-mechanical delay between delivery of a pacing pulse at the RV and detection of a mechanical contraction by the IP lead is determined during implant and periodically thereafter. The electro-mechanical delays are monitored for a sudden change indicative of intrapericardial lead migration. In one arrangement, a sudden change corresponds to an approximate 20-30% change in electro-mechanical delay between adjacent observed electro-mechanical delays. More gradual changes in electro-mechanical delays, for example, a 20-30% change that occurs over a period of weeks or months, may be indicative of heart defect or heart failure progression.

In order to monitor heart failure status or intrapericardial lead stability it may be desirable for a patient to undergo automated periodic tests (such as daily) to train the trends and to separate the changes from heart failure progression and intrapericardial lead migration. For example electrical separation Δ can vary due to reverse remodeling (either dimension of left ventricle change and conduction velocity) but changes are expected to occur slowly over a period of weeks or months. Sudden changes, such as those that occur from one day to the next, in electrical separation, pacing latency or evoked response, cardiogenic impedance, and/or electro-mechanical delays, etc. would indicate intrapericardial lead dislocation. A running average can also be used to detect sudden changes in time. The values with slow changes in time can be used to monitor heart failure progressions. Additionally, data can be analyzed in the frequency domain to determine if the change in data occurred slowly and is due to heart failure progression, or suddenly and is due to lead movement.

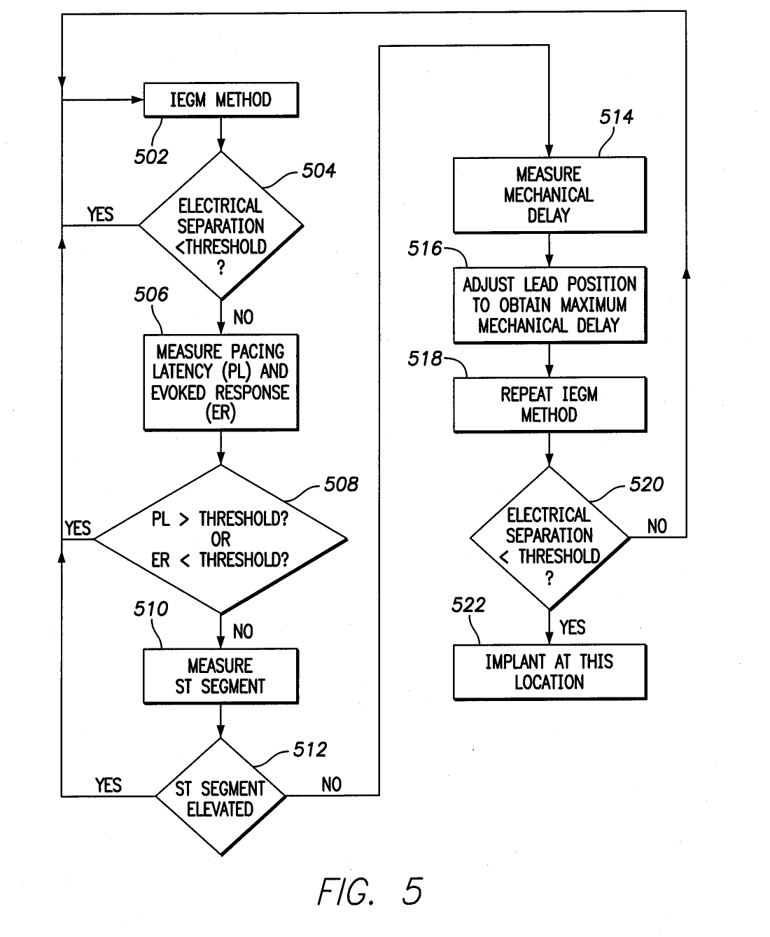

FIG. 5 illustrates an exemplary flowchart of a method for guiding (during implant) and monitoring (after implant) the position of an intrapericardial lead position. The process may be performed by a controller device such as the microcontroller 60 of the implantable medical device 10 and/or a controller device associated with the external heart monitoring device 406. At block 502, the IEGM guided method described above is implemented to determine a desired location of the intrapericardial lead 402, i.e., IEGM based time delays derived from atrio-ventricular, intra-ventricular and/or inter-ventricular intrinsic conduction delays are determined. At block 504, it is determined if the time delay (or electrical separation) satisfies a timing threshold criteria. For example, in the case of timing delays based on atrio-ventricular delay and/or intra-ventricular delay, the threshold value may be approximately 30 milliseconds. Alternatively, the threshold may be a range of values, for example between approximately 30 ms and approximately 160 ms, and a time delay that falls within that range is considered indicative of proper IP lead location. In the case where the timing delay is an inter-ventricular conduction delay, the threshold may be approximately 80 ms, wherein a time delay of at least 80 ms is indicative of an appropriate IP lead location.

If the time delay is below the threshold value, the IP lead location is deemed to be unsuitable, and the process returns to block 502 for re-positioning of the lead. Otherwise, the IP lead location is deemed to be proper and the process proceeds to determine if the IP lead is placed at a slow conduction zone, e.g., ischemic or infract region. One or more of a number of different approaches for detecting a slow conduction zone may be employed. For example, at block 506 the pacing latency (the time delay between pacing stimulus delivery and evoked response detection at the pacing site) and/or evoked response magnitude are measured with respect to the IP lead. At block 508, it is determined whether the pacing latency and/or evoked response meet their respective threshold value, as described above. For example, if either the pacing latency is greater than the threshold value or the evoked response is less than the threshold value then the associated site or location may be considered an ischemic or an infarct zone, in which case, the process returns to block 502 to use the IEGM method to find a different appropriate intrapericardial lead location.

At block 510, another approach for detecting a slow conduction zone relies on analysis of electrocardiogram signals. As described above, assessing an ST segment shift can verify whether a location of a lead representing an increased electrical delay includes non-ischemic, non-infracted tissue. At block 510 an ST segment is measured. At block 512, it is determined whether the ST segment is elevated. If the ST segment is elevated, an ischemia or infarct region could be in between the sites, and another site should be selected as the process returns to block 502.

If no ischemic or infarct regions are detected, the process continues to block 514 where mechanical delay is measured in order to optimize the determined intrapericardial lead. This process involves obtaining a mechanical delay measure for the determined intrapericardial lead location and each of a plurality of alternate intrapericardial lead positions in the region of the IP lead location, as previously determined by the IEGM method, and placing the IP lead at the location having the greatest mechanical delay (block 516). In one arrangement the mechanical delay is the time delay between an intrinsic atrial depolarization and a mechanical contraction sensed by a mechanical sensor on the IP lead. In another arrangement, the mechanical delay measure is the time delay between delivery of a pacing pulse to the RV and a mechanical contraction sensed at the IP lead.

Once the IP lead is positioned based on mechanical delay, the process continues to block 518 where the IEGM method is repeated to verify, at block 520, that the electrical separation at the current location of the IP lead satisfies the electrical separation threshold. If the electrical separation does not satisfy the threshold the process returns to block 502 to select another appropriate location for the IP lead. Otherwise, the process ends at block 522 where the intrapericardial lead 402 is implanted at the current location having both an appropriate electrical separation and mechanical delay.

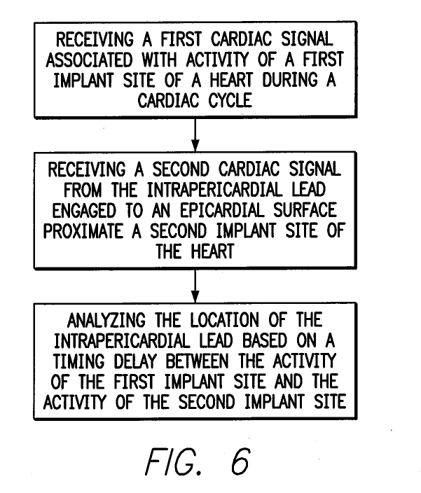

FIG. 6 illustrates flowchart of a method for guiding (during implant) and monitoring (after implant) a location of an intrapericardial lead according to aspects of the disclosure. At block 602, the method starts with receiving a first cardiac signal associated with activity of a first implant site of a heart during a cardiac cycle. At block 604, the method includes receiving a second cardiac signal from the intrapericardial lead engaged to an epicardial surface proximate a second implant site of the heart. The second cardiac signal can be associated with activity of the second implant site of the heart during the cardiac cycle. At block 606, the method further includes analyzing the location of the intrapericardial lead based on a timing delay between the activity of the first implant site and the activity of the second implant site.

In one configuration, the apparatus for guiding and monitoring a location of an intrapericardial lead includes a means for receiving a first cardiac signal associated with activity of a first implant site of a heart during a cardiac cycle. In one aspect of the disclosure, the first cardiac signal receiving means may be the microcontroller 60, the lead location system 400, the intrapericardial lead 402, the implantable medical device 404, the heart chamber lead 408, the external heart monitoring device 406 and/or the lead location module 122 configured to perform the functions recited by the first cardiac signal receiving means. The apparatus is also configured to include a means for receiving a second cardiac signal from the intrapericardial lead engaged to an epicardial surface proximate a second implant site of the heart. In one aspect of the disclosure, the second cardiac signal receiving means may be the microcontroller 60, the lead location system 400, the intrapericardial lead 402, the implantable medical device 404, the heart chamber lead 408, the external heart monitoring device 406 and/or the lead location module 122 configured to perform the functions recited by the second cardiac signal receiving means. The apparatus is also configured to include a means for analyzing the location of the intrapericardial lead based on a timing delay between the activity of the first implant site and the activity of the second implant site. In one aspect of the disclosure, the analyzing means may be the microcontroller 60, the lead location system 400, the implantable medical device 404, the external heart monitoring device 406 and/or the lead location module 122 configured to perform the functions recited by the analyzing means.

The methodologies described herein may be implemented by various means depending upon the application. For example, these methodologies may be implemented in hardware, firmware, software, or any combination thereof. For a hardware implementation, the processing units, including programmable microcontroller 60 may be implemented within one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, electronic devices, other electronic units designed to perform the functions described herein, or a combination thereof.

For a firmware and/or software implementation, the methodologies may be implemented with modules (e.g., procedures, functions, and so on) that perform the functions described herein. Any machine or computer readable medium tangibly embodying instructions that may be in a form implantable or coupled to an implantable medical device may be used in implementing the methodologies described herein. For example, software code may be stored in a memory and executed by a processor. When executed by the processor, the executing software code generates the operational environment that implements the various methodologies and functionalities of the different aspects of the teachings presented herein. Memory may be implemented within the processor or external to the processor. As used herein the term “memory” refers to any type of long term, short term, volatile, nonvolatile, or other memory and is not to be limited to any particular type of memory or number of memories, or type of media upon which memory is stored.

Although the present teachings and its advantages have been described in detail, it should be understood that various changes, substitutions and alterations can be made herein without departing from the spirit and scope of the present teachings as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present teachings, processes, machines, manufacture, compositions of matter, means, methods, or steps, presently existing or later to be developed that perform substantially the same function or achieve substantially the same result as the corresponding embodiments described herein may be utilized according to the present teachings. Accordingly, the appended claims are intended to include within their scope such processes, machines, manufacture, compositions of matter, means, methods, or steps.

What is claimed is:

1. A method for guiding and/or monitoring a location of an intrapericardial lead, comprising:
sensing a first cardiac signal associated with an activity of a first implant site of a heart during a cardiac cycle;
sensing a second cardiac signal from the intrapericardial lead located on an epicardial surface proximate a second implant site of the heart, the second cardiac signal associated with an activity of the second implant site of the heart during the cardiac cycle;
obtaining a timing delay between the activity of the first implant site and the activity of the second implant site; and
analyzing the location of the intrapericardial lead based on the timing delay
repeating the preceding steps until an appropriate intrapericardial lead location is determined.

2. The method of claim 1 wherein the activity of the first implant sight is a cardiac polarization, the activity of the second implant sight is a cardiac polarization and analyzing comprises comparing the timing delay (electrical separation) to a threshold indicative of an appropriate intrapericardial lead location.

3. The method of claim 2 wherein the first implant sight is the right ventricle and the second implant site is the left ventricle, and the timing delay is obtained by:
determining a delay between a stimulation pulse delivered to the atrium and the cardiac polarization sensed at the right ventricle (AR_{RV});
determining a delay between the stimulation pulse delivered to the atrium and the cardiac polarization sensed at the left ventricle (AR_{LV}); and
calculating the difference between the AR_{RV} and AR_{LV}.

4. The method of claim 3 wherein the threshold is a range of values between approximately 30 ms and approximately 160 ms and a time delay within the range is indicative of appropriate intrapericardial lead location.

5. The method of claim 2 wherein the first implant sight is the right ventricle and the second implant site is the left ventricle, and the timing delay is obtained by:
determining a time of an intrinsic cardiac polarization sensed at the right ventricle (R_{RV});
determining a time of an intrinsic cardiac polarization sensed at the left ventricle (R_{LV}); and
calculating the difference between the R_{RV} and R_{LV}.

6. The method of claim 5 wherein the threshold is a range of values between approximately 30 ms and approximately 160 ms and a time delay within the range is indicative of appropriate intrapericardial lead location.

7. The method of claim 2 wherein the first implant sight is the right ventricle and the second implant site is the left ventricle, and the timing delay is obtained by:
determining one of a delay between a stimulation pulse delivered to the right ventricle and the cardiac polarization sensed at the left ventricle (RV_{pace}LVₛₑₙₛₑ), and a delay between a stimulation pulse delivered to the left ventricle and the cardiac polarization sensed at the right ventricle (LV_{pace}RVₛₑₙₛₑ).

8. The method of claim 7 wherein the threshold is approximately 80 ms and a time delay of at least 80 ms is indicative of an appropriate intrapericardial lead location.

9. The method of claim 2 wherein the first cardiac signal and the second cardiac signal are electrograms.

10. The method of claim 1 further comprising determining whether the intrapericardial lead location is at or near an ischemic region or myocardial infarct zone.

11. The method of claim 10 wherein determining whether the intrapericardial lead location is at or near an ischemic region or myocardial infarct zone comprises:
delivering a pacing stimulation pulse to a pacing site through the intrapericardial lead;
detecting for an evoked response at the pacing site;
calculating the pacing latency as the difference between the time of delivery and the time of detection; and
comparing the pacing latency to a threshold indicative of an ischemic or infarct zone

12. The method of claim 11 wherein the threshold is approximately 70 ms and a pacing latency greater than the threshold is indicative of an ischemic or infarct zone.

13. The method of claim 10 wherein determining whether the intrapericardial lead location is at or near an ischemic region or myocardial infarct zone comprises:
delivering a pacing stimulation pulse to a pacing site through the intrapericardial lead;
measuring a parameter of an evoked response sensed at the pacing site; and
comparing the parameter to a threshold indicative of an ischemic or infarct zone.

14. The method of claim 13 wherein the parameter is a peak amplitude of the evoked response, the threshold is approximately 5 mV and a peak amplitude less than the threshold is indicative of an ischemic or infarct zone.

15. The method of claim 13 wherein the parameter is a slope of the evoked response, the threshold is approximately 0.3 and a slope less than the threshold is indicative of an ischemic or infarct zone.

16. The method of claim 10 wherein determining whether the intrapericardial lead location is at or near an ischemic region or myocardial infarct zone comprises:
sensing a cardiac electrogram between the first implant site and the second implant site;
determining a deviation between the baseline ST segment elevation and the ST segment elevation in the cardiac electrogram and a baseline ST segment elevation; and
comparing the deviation in ST segment elevation to a threshold indicative of an ischemic or infarct zone.

17. The method of claim 1 further comprising optimizing the determined intrapericardial lead location by:
obtaining a mechanical delay measure for the determined intrapericardial lead location and each of a plurality of alternate intrapericardial lead positions in the region of the determined intrapericardial lead location;
placing the intrapericardial lead at the location having the greatest mechanical delay.

18. The method of claim 17 wherein the intrapericardial lead comprises a mechanical movement sensor and the mechanical delay measure comprises the time delay between an intrinsic atrial depolarization and a mechanical contraction sensed at the second implant site by the mechanical sensor.

19. The method of claim 17 wherein the intrapericardial lead comprises a mechanical movement sensor and the mechanical delay measure comprises the time delay between delivery of a pacing pulse to the first implant site and a mechanical contraction sensed at the second implant site by the mechanical sensor.

20. The method of claim 17 further comprising repeating the sensing, obtaining and analyzing at the location having the greatest mechanical delay to verify that the location has a time delay indicative of an appropriate intrapericardial lead location.

21. The method of claim 1 further comprising determining whether the intrapericardial lead has migrated.

22. The method of claim 21 wherein the activity of the first implant sight is a cardiac polarization, the activity of the second implant sight is a cardiac polarization, and determining whether the intrapericardial lead has migrated comprises:
periodically determining an observed timing delay between the first cardiac signal and the second cardiac signal after implant of the intrapericardial lead; and
monitoring the observed timing delays for a sudden change indicative of intrapericardial lead migration.

23. The method of claim 22 wherein a sudden change corresponds to a difference in timing delay of approximately 30 ms between adjacent observed timing delays.

24. The method of claim 21 wherein determining whether the intrapericardial lead has migrated comprises:
determining a baseline pacing threshold at the second implant site of the heart during implant of the intrapericardial lead;
periodically determining an observed pacing threshold at the second implant site of the heart after determining the baseline pacing threshold;
monitoring the observed pacing thresholds for a sudden change indicative of intrapericardial lead migration.

25. The method of claim 24 wherein a sudden change corresponds to a difference in pacing thresholds of approximately 1 volt between the baseline pacing threshold and an observed pacing threshold.

26. The method of claim 21 wherein determining whether the intrapericardial lead has migrated comprises:
periodically determining observed cardiogenic impedances using the intrapericardial lead after implant of the intrapericardial lead; and
monitoring the observed cardiogenic impedances for a sudden change indicative of intrapericardial lead migration.

27. The method of claim 26 wherein the observed cardiogenic impedances comprise a stroke impedance (SZ) value corresponding to a difference between a maximum impedance measurement and a minimum impedance measurement obtained during a cardia cycle, and a point impedance (Z) corresponding to an impedance measurement obtain at a particular point of the cardiac cycle, and a sudden change corresponds to an approximate 20% change in the ratio ΔZ/SZ, where ΔZ is the difference between adjacent daily impedance values.

28. The method of claim 21 wherein the activity of the first implant sight is a cardiac polarization, the activity of the second implant sight is a cardiac contraction and determining whether the intrapericardial lead has migrated comprises:
determining an electro-mechanical delay between the first cardiac signal and the second cardiac signal during implant of the intrapericardial lead;
periodically determining an observed electro-mechanical delay between the first cardiac signal and the second cardiac signal after implant of the intrapericardial lead; and
monitoring the observed electro-mechanical delays for a sudden change indicative of intrapericardial lead migration.

29. The method of claim 28 wherein a sudden change corresponds to an approximate 20-30% change in electro-mechanical delay between adjacent observed electro-mechanical delays.

30. The method of claim 1 further comprising monitoring heart failure status based on changes over time in one of timing delay, pacing latency, evoked response, cardiogenic impedance and mechanical delay measured using the intrapericardial lead.

31. An apparatus for guiding and/or monitoring a location of an intrapericardial lead, comprising:
a memory; and
at least one processor coupled to the memory and configured to:
sense a first cardiac signal associated with an activity of a first implant site of a heart during a cardiac cycle;
sense a second cardiac signal from the intrapericardial lead located on an epicardial surface proximate a second implant site of the heart, the second cardiac signal associated with an activity of the second implant site of the heart during the cardiac cycle;
obtain a timing delay between the activity of the first implant site and the activity of the second implant site; and
analyze the location of the intrapericardial lead based on the timing delay
repeating the preceding steps until an appropriate intrapericardial lead location is determined.
